(19) United States
(12) Patent Application Publication
(10) Pub. No.: US 2013/0315650 A1
(43) Pub. Date: Nov. 28, 2013

(54) COSMETIC COMPOSITION FOR FACE AND THE AREA AROUND THE EYES

(71) Applicants: Guillaume Cassin, Villebon Sur Yvette (FR); Myriam Kauffmann, Ivry Sur Seine (FR)

(72) Inventors: Guillaume Cassin, Villebon Sur Yvette (FR); Myriam Kauffmann, Ivry Sur Seine (FR)

(73) Assignee: L'OREAL, Paris (FR)

(21) Appl. No.: 13/988,242

(22) PCT Filed: Nov. 17, 2011
(86) PCT No.: PCT/EP2011/070394

§ 371 Date: Aug. 1, 2013

Related U.S. Application Data
(60) Provisional application No. 61/416,374, filed on Nov. 23, 2010.

(30) Foreign Application Priority Data
Nov. 17, 2010 (FR) 1059436

(51) Int. Cl. A61K 8/02; A61Q 1/10; A45D 40/26

(52) U.S. Cl. CPC A61K 8/0245; A45D 40/26; A45D 40/261; A45D 40/262; A61Q 1/10
USPC 401/196; 401/208; 401/268; 424/401

(57) ABSTRACT

The present invention relates to a cosmetic composition for caring for and/or making up keratin materials, in particular the skin of the face, comprising, in a physiologically acceptable medium, (i) at least one pigment in the form of a porous or non-porous, spherical or non-spherical, mineral or organic white particle having a refractive index of greater than or equal to 1.6 and a size by volume of preferably less than 30 μm, and (ii) at least one filler in the form of a porous or non-porous, spherical or non-spherical particle having a refractive index of less than or equal to 2 and a size by volume of preferably less than 30 μm, which is distinct from said pigments (i), and characterized in that the total content of filler(s) is less than or equal to 4% by weight, relative to the total weight of the composition.

The present invention relates to a cosmetic composition comprising “white” pigments and “scattering” fillers, in particular for the area around the eyes and intended in particular for reducing the visibility of dark circles.

The anatomical region of the area around the eyes exhibits such morphological, histological and physiological differences with the rest of the face that, in order to be treated effectively and with subtlety, both in terms of care and in terms of makeup, it requires formulae and methods which take these specificities into account.

Indeed, the eye of the observer perceives at the same time and transmits to the brain information of various natures: 3-dimensional (3D) macromorphology, microrelief, and colours, in particular their location and their distribution.

As regards the 3D-macromorphology, the region of the area around the eye comprises surfaces that are relatively flat and parallel to the plane of the face, or to the front of the forehead, but also convex regions, some of which are mobile, such as the part of the eyelids which covers the eyeball (tarsal part) and which can be more or less creased according to their open or closed position, concave regions which are inclined as regards the part which is superposed on the sides of the eye sockets, and regions that are inclined almost perpendicular to the face as regards the regions close to the inner angles or canthi of the eyes (or medial commissure, located at the level of the upper part of the wings of the nose), skin folds which follow the free edge of the eyelids, called the orbito-palpebral sulcus, which are accentuated when the eye is open, with various “complications” such as opening of the tear ducts at the level of the medial commissures, sometimes forming a clearly visible caruncle, or in the form of a cord as regards the edge on which the eyelashes are implanted.

Moreover on the upper eyelids can sit one or two horizontal creases, more or less parallel to their free border, with a lot of individual and ethnic variants. As an example, approximatively half of all Asians do not have this crease and most of Asians have some form of medial canthal fold (from Chen and Khan Saunders Elsevier 2010). These creases and folds evolve with aging, in particular with volume loss, slackening. These macro-relief characteristics (too much or not enough marked) need for specific solutions that permits smoothening their visibility, in particular for Asians skins and aged skins.

With regard to the microrelief, the eyelids may be smooth, crumpled, have fine lines or even wrinkles running through them, and, on the part close to the eye, uncover the opening of various glands.

Finally with regard to the colour, under the lightings encountered in usual life circumstances, it never appears completely homogeneous to the observer owing to the shadows cast by the relief (the nose, the convexities, the edges of the concavities) and the microrelief, and also the local heterogeneities in terms of pigment, vascularization and transparency of the skin.

There are in particular in many individuals more or less extensive regions of very varying shapes which are relatively delimited or of gradual intensity (“in shading”), that have a more red, more bluish or purplish, more brown or more grey intrinsic hue than the complexion of the adjacent regions, the cheekbones or the temples, or appear darker than the regions that surround them. This is what contributes to the notion of dark circles, and can be amplified by the shadows cast which were described above.

The prior art describes the use of composite platelet pigments (for example: mica/titanium, mica/titanium/iron oxide, mica/titanium/silica, mica/titanium/tin, etc.) having a size of less than 25 μm and of transparent pigments in compositions for correcting skin dyschromia (WO2004/045524); or else the use of interference pigments (WO01/51017, U.S. Pat. No. 5,690,916) and/or of metal oxides (US2005079190) or composite particles containing metal oxides (EP 1 785 129) for correcting dyschromia.

However, the prior art compositions are not entirely satisfactory; this is because they can only minimize some of these problems (macromorphology, colour, microrelief) and sometimes at the cost of the amplification of another.

### Thus:

a covering formula containing metal oxide pigments and/or high amounts of fillers cannot provide satisfaction since it will mask the colour but may amplify the shadows cast and will require the application of a foundation in order to avoid the visibility of the “touch up”. It will given an artificial or set appearance in particular on the mobile and expressive regions. It is also necessary, in the case of coloured formulae, for the formula to have the same shade as the complexion of the individual, which means developing as many colours as there are skin shades;
a formula containing composite interference pigments or only fatty substances will be shiny and will not be able to provide satisfaction since it will reduce the shadows but, on the other hand, will accentuate the defects or microdefects in relief, such as the visibility of wrinkles, of creases, of fine lines, of withering, or in the contrary will increase paying attention to a lack of relief;
a transparent formula will be able to treat the skin tissue and the physiology of the cells more deeply, but will in no way be able to minimize the visibility of local defects; it may even accentuate them if it makes the skin temporarily more transparent.

There remains therefore the need to have a specific composition for the area around the eyes which makes it possible by itself to attain a significant decrease in the visibility of the dark circles while at the same time maintaining a natural “bare skin” appearance.

The present invention aims precisely to propose, in order to correct the appearances considered to be unsightly, compositions which are not at all very covering, not very coloured and which do not give the skin a shiny or opaque and artificial appearance. In addition, they are polyvalent and are readily suitable for varied skins since it is not necessary for the shade of the formula to be exactly the same as the shade of the complexion of the individuals.

They make it possible to combine components with a mechanical effect, such as tensioning agents, smoothing agents or firming agents which may also improve optionally superposed makeup. Finally, they make it possible to easily transport biological active agents for all the compartments of the skin or of the mucous membranes, such as components with a moisturizing, anti-aging, anti-inflammatory, antioxidant, anti-glycation, pigmentary, blood or lymphatic microcirculatory, or anti-allergic purpose, or which are able to facilitate protection against UV-induced damage or the repair of said damage, and also to improve local sensations and comfort.

The compositions of the invention therefore make it possible to reduce the visibility of dark circles without modifying the natural complexion and while keeping a bare skin appearance, making it possible to be free of the use of foundation.

These compositions are based on the combination of a “white” pigment in particular in a small amount and of “scattering” fillers, which results in what is in common language called a desaturation of the colour or decrease in the chroma C*.

According to one preferred embodiment of the invention, the content of white pigment(s) according to the invention will range from 0.1 to 4% by weight, relative to the total weight of the composition.

The chroma C* is defined by the CIE, International Commission on Illumination, and expressed by the equation

C*=\sqrt{a*₂+b*²},

a* and b* representing the colorimetric coordinates in the system L*a*b* (red/green axis for a* and yellow/blue axis for b*) or L*C*h.

The correlation of (a*,b*) with (C*, h) is given by

$${C}^{*} = \sqrt{{a}^{* 2} + {b}^{* 2}} , \\ h = arctan \frac{{b}^{*}}{{a}^{*}}$$

In the rest of the description, reference will be made indifferently to chroma or saturation C* or C.

In addition, in the rest of the description, reference will be made indifferently to luminance or lightness L* or L.

The compositions according to the invention do not require the addition of fillers in a content greater than 4% by weight, nor of iron oxides in a content greater than 0.5% by weight, as in the prior art compositions.

Once spread into a thin film and dried according to the protocol described hereinafter, the compositions of the invention can in particular be characterized by the following optical properties:

−25<ΔC<−2 where C is the chroma
5<ΔL<30 where L is the luminance
50<% Tr<70, in particular 50<% Tr<68, or even 50<% Tr<66 where % Tr is the transparency,
according to the protocol described hereinafter.

The present invention therefore relates to a cosmetic composition for caring for and/or making up keratin materials, in particular the skin of the face and especially of the area around the eye, comprising, in a physiologically acceptable medium,
(i) at least one pigment in the form of a porous or non-porous, spherical or non-spherical, mineral or organic white particle having a refractive index of greater than or equal to 1.6 and a size by volume of preferably less than 30 μm, and
(ii) at least one filler (otherwise referred to as “scattering” filler) in the form of a porous or non-porous, spherical or non-spherical particle having a refractive index of less than or equal to 2 and a size by volume of preferably less than 30 μm, which is distinct from said pigments (i),
and characterized in that the total content of filler(s) is less than or equal to 4% by weight, relative to the total weight of the composition.

Preferably, the said filler has a size by volume of more than 500 nm, preferably of more than 1 μm.

Preferably, the filler is a matifying filler as defined hereunder.

According to one preferred embodiment, the content of “white” pigments (i) is also less than or equal to 4% by weight, relative to the total weight of the composition.

According to one particular embodiment, the “scattering” fillers (ii)/“white” pigments (i) weight ratio ranges from 0.1 to 2.5.

The invention also relates to a cosmetic composition for caring for and/or making up keratin materials, in particular the skin of the face and especially of the area around the eye, comprising, in a physiologically acceptable medium,
(i) at least one pigment in the form of a porous or non-porous, spherical or non-spherical, mineral or organic white particle having a refractive index of greater than or equal to 1.6 and a size by volume of preferably less than 30 μm, and
(ii) at least one filler in the form of a porous or non-porous, spherical or non-spherical particle having a refractive index of less than or equal to 2 and a size by volume of preferably less than 30 μm, which is distinct from said pigments (i),
and characterized in that it has the following optical properties:
−25<ΔC<−2 where C is the chroma
5<ΔL<30 where L is the luminance
50<% Tr<70, in particular 50<% Tr<68, or even 50<% Tr<66 where % Tr is the transparency,
measured in accordance with the protocol described hereinafter.

Preferably, the said filler has a size by volume of more than 500 nm, preferably of more than 1 μm.

Preferably, the filler is a matifying filler as defined hereunder.

The invention also relates to a cosmetic device for packaging and applying a composition according to the invention, in particular in the form of a pen, comprising at least
a) a reservoir comprising a composition according to the invention, and
b) an applicator in particular chosen from a brush, a perforated flexible blade, a felt tip, a sponge, a roller, a ball, a plate bearing several balls, a pad, or a head generating physical, mechanical (vibrating), thermal (hot or cold), electrical or electromagnetic (light) pulses.

The invention also relates to a cosmetic process for caring for and/or making up keratin materials, in particular the skin, comprising the application of a composition according to the invention to the skin, in particular of the face.

According to one particular and preferred embodiment, the composition according to the invention is applied to the area around the eye, in particular to the dark circles or bags, so as to reduce the visibility thereof.

In a particular embodiment, the composition is applied to Asians skins or aged skins, in particular to the face and preferably to the area around the eye.

### “White” Pigments

The particular pigment(s) used in the composition according to the invention denotes a mineral or organic particle which is insoluble in an aqueous solution, said particle being white, spherical or non-spherical and porous or non-porous, and having a refractive index of greater than or equal to 1.6, in particular greater than or equal to 1.8, preferably ranging from 1.6 to 2.5.

The “pigments” according to the invention have a size by volume of preferably less than 30 μm, in particular less than 15 μm.

In particular, the “pigments” according to the invention have a size by volume of more than 500 nm, preferably more than 1 μm, and preferably less than 30 μm, in particular less than 15 μm.

In particular, said pigments are in the form of a white powder.

The term “white” is intended to mean in particular white and derivatives thereof (off-white, alabaster white, silver white, etc.) or with a silvery, white tint, as opposed to primary colours and derivatives.

In particular, the “white” pigments have a luminance value L close to 100 in the TSL (tint, saturation, luminance) system.

The colorimetric measurements L* and C* in the CIE Lab 1976 colorimetric space may be performed using a MINOLTA CR400® colorimetric chromameter.

To do this, the pigment is pre-dispersed (grinded with tricylinder) in a transparent cosmetic medium in a % able to confere opacity (ex: 5-6% of pigment) and then introduced into a 15 ml jar (aperture diameter: 1,9 cm; depth: 1,8 cm). The surface of the cosmetic medium introduced is smoothed out by flattening with a glass slide. The colorimeter cell is then placed in contact with this surface and the colorimetric parameters are determined.

The MINOLTA CR400® colorimetric chromameter is calibrated with a white reference (white ceramic for calibration such as Spectralon) with lighthness L* value of 100 and saturation C* value of 0, and a trap light.

According to a particular embodiment, the ‘white’ pigments of the invention will have in particular lightness L* value superior or equal to 95 and saturation C* value inferior or equal to 3 according to the previous disclosed protocol.

Thus, the composition of the invention comprises at least one pigment in the form of a porous or non-porous, spherical or non-spherical, mineral or organic white particle having a refractive index of greater than or equal to 1.6 and a size by volume of preferably less than 30 μm, in particular size by volume of more than 500 nm, preferably of more than 1 μm, and less than 30 μm.

Said pigments (i) are in particular chosen from titanium oxides, zinc oxides, optionally dispersed bismuth oxychloride, insoluble barium salts, in particular barium sulphate, calcium carbonate, calcium sulphate, plaster, natural powders of mineral, animal or vegetable origin, chalk powder, gypsum powder, alabaster powder or selenite powder, egg-shell or shellfish powders, vegetable ivory, and mixtures thereof.

Bismuth oxychloride can be used as it is (in powder form) or advantageously in the form of a dispersion in an ester oil, chosen in particular from isodecyl neopentanoate; isocetyl octanoate; isononyl isononanoate, isopropyl isostearate, 2-ethylhexyl hydroxystearate, 2-octyldodecyl stearate, isostearyl isostearate; and mixtures thereof. Preferably, the dispersion of bismuth oxychloride comprises 2-ethylhexyl hydroxystearate, such as the product sold under the name Biron® Liquid Silver by the company MERCK.

According to one alternative, use may also be made, as white pigments (i), of white-tinted interference pigments, such as fluorphlogopite-based composite particles coated with titanium oxide and with tin oxide, for instance those sold under the name SYNCRYSTAL SILVER by the company ECKART, which are distinct from interference pigments with a non-white coloured tint, such as, in particular, the Timiron Silk Blue mica/titanium oxide/tin oxide blue-tinted pearlescent agents.

According to one particular embodiment, said pigments (i) are white-tinted interference pigments.

According to another particular embodiment, said pigments (i) are chosen from titanium oxides, zinc oxides, optionally dispersed bismuth oxychloride, insoluble barium salts, in particular barium sulphate, calcium carbonate, calcium sulphate, plaster, natural powders of mineral, animal or vegetable origin, chalk powder, gypsum powder, alabaster powder or selenite powder, egg-shell or shellfish powders, vegetable ivory and mixtures thereof, preferably from titanium oxides, zinc oxides and mixtures thereof, and are not interference pigments.

According to a particular embodiment, the composition comprises as pigments (i), at least titanium oxides and optionnally in association white-tinted interference pigments.

Not included in the definition of pigments (i) according to the invention are titanium oxides encapsulated in silica particles, such as particles of porous silica comprising titanium dioxides, in particular sold under the name God Ball PC-LS by the company Suzuki Oil and Fat or PC-LS-14 by MIYOSHI KASEI; such particles are additional fillers according to the invention, as described hereinafter.

Preferably, the pigments (i) are chosen from coated or uncoated titanium oxides of rutile or anatase form.

Thus, according to one particular embodiment, the composition according to the invention comprises at least coated or uncoated titanium oxides of rutile or anatase form.

The pigments used according to the invention may or may not be totally or partially surface-treated with at least one hydrophilic or hydrophobic treatment agent.

For the purpose of the invention, the surface treatment of a pigment according to the invention generally denotes total or partial surface treatment with a surface agent, absorbed, adsorbed or grafted onto said pigment.

The surface-treated pigments can be prepared according to techniques for surface treatment of chemical, electronic, mechanochemical or mechanical nature that are well known to those skilled in the art. Commercial products may also be used.

The surface agent may be absorbed, adsorbed or grafted onto the pigments by solvent evaporation, chemical reaction or creation of a covalent bond.

The surface treatment can represent from 0.1 to 50% by weight, and in particular from 0.5 to 5% by weight, of the total weight of the coated pigment.

The surface treatment can be carried out, for example, by adsorption of a liquid surface agent at the surface of the solid particles by simple mixing with stirring of the pigments and of said surface agent, optionally under hot conditions, prior to the incorporation of said pigments into the other ingredients of the makeup or care composition.

The surface treatment can be carried out, for example, by chemical reaction of a surface agent with the surface of the pigments and creation of a covalent bond between the surface agent and the particles. This method is in particular described in U.S. Pat. No. 4,578,266.

The chemical surface treatment can consist in diluting the surface agent in a volatile solvent, in dispersing the pigments in this mixture, and then in slowly evaporating the volatile solvent, such that the surface agent is deposited at the surface of the pigments. This volatile solvent may be water.

### Lipophilic or Hydrophobic Treatment Agent

According to one particular embodiment of the invention, the pigments according to the invention can be surface-treated with at least one hydrophobic or lipophilic treatment agent chosen from silicone surface agents; fluorinated surface agents; fluorosilicone surface agents; metal soaps, N-acylated amino acids or salts thereof; lecithin and derivatives thereof; isopropyl triisostearyl titanate; isostearyl sebacate; vegetable or animal natural waxes, polar synthetic waxes; fatty esters; fatty alcohols; phospholipids, and mixtures thereof.

According to one particular embodiment, the pigments are surface-treated with N-acylated amino acids or salts thereof. The N-acylated amino acids can comprise an acyl group containing from 8 to 22 carbon atoms, for instance a 2-ethylhexanoyl, caproyl, lauroyl, myristoyl, palmitoyl, stearoyl or cocoyl group. The salts of these compounds may be the aluminium, magnesium, calcium, zirconium, zinc, sodium or potassium salts. The amino acid may, for example, be lysine, glutamic acid or alanine. In particular, it will be lauroyllysine.

### Hydrophilic Treatment Agent

According to one particular embodiment, the pigments may be surface-treated with at least one hydrophilic treatment agent chosen from biological polymers, carbohydrates, polysaccharides, polyacrylates or polyethylene glycol derivatives. Mention may also be made of mineral agents such as silica, silicates, alumina and mixtures thereof (for example: silica/alumina).

By way of examples of biological polymers for coating the materials to be solubilized according to the invention, mention may be made of polymers based on monomers of carbohydrate type, in particular those derived from algae, from land plants, from fungi or from biotechnologies, from plankton or from arthropod shells. More particularly, mention may be made of guar gum, locust bean gum, xanthan gum, gum arabic, sclerotium gum; konjac, carrageenans, alginates and derivatives thereof, pectins, agar-agar; glycogen; dextran; starch and derivatives thereof; cellulose and derivatives thereof; hyaluronic acid salts, such as sodium hyaluronate, and derivatives thereof; soluble proteoglycans; glycosaminoglycans, chitins, chitosans and derivatives thereof, and mixtures thereof. C₁-C₂₀ glycol alkylenes or C₁-C₂₀ glycol alkylene ethers, alone or used in combination with tri-C₁-C₂₀-alkylsilanes, can also be used as surface-treatment agents. By way of examples, mention may be made of pigments surface-treated with PEG alkyl ether alkoxysilane, for instance the pigments treated with PEG-8-methyl ether triethoxysilane sold by the company KOBO under the name “SW” pigments.

The pigments (i) preferentially used are coated or uncoated, encapsulated or non-encapsulated titanium dioxides TiO₂ of rutile or anatase form.

Mention may also be made of zinc oxides.

According to one preferred embodiment, the composition of the invention comprises at least titanium oxides, in particular TiO₂ which are surface-treated with alumina/silica/trimethylolpropane, sold under the names Tipaque PF671 from Ishihara Sangyo and SA-TAO from Myoshi Kasei.

According to another preferred embodiment, the composition of the invention comprises at least one bismuth oxychloride optionally dispersed in 2-ethylhexyl hydroxystearate, such as the product sold under the name Biron® Liquid Silver by the company MERCK.

According to another embodiment, the composition of the invention comprises at least fluorphlogopite-based composite particles coated with titanium oxide and with tin oxide, such as those sold under the name SYNCRYSTAL SILVER by the company ECKART.

Said pigments (i) are generally present in a composition of the invention in a content ranging from 0.1% to 4% by weight, preferably from 0.5 to 3% by weight (of active material for the case where the pigment is dispersed in a vehicle or coated), relative to the total weight of said composition.

In the case of white-tinted interference pigments (i), as is the case of the fluorphlogopite-based composite particles coated with titanium oxide and with tin oxide mentioned above, they will preferably be present in the composition in an amount of less than or equal to 2% by weight, especially less than or equal to 1% by weight, in particular less than or equal to 0.5% by weight, relative to the total weight of said composition.

### Fillers (Termed “Scattering” Fillers)

For the purpose of the invention, the term “scattering filler” denotes a porous or non-porous, hollow or non-hollow, spherical or non-spherical particle having a refractive index of less than or equal to 2, in particular less than or equal to 1.8, preferably ranging from 1.3 to 1.6, which is distinct from the pigments (i) described above. The “scattering fillers” according to the invention preferably have a size by volume of less than 30 μm, preferentially less than 15 μm.

In particular the “scattering filler” of the invention have a size by volume of more than 500 nm, preferably of more than 1 μm and less than 30 μm, preferably less than 15 μm.

In one preferential embodiment of the invention, the “scattering fillers” are spherical.

In one preferential embodiment of the invention, the “scattering fillers” are porous.

In this case, the specific surface area of the particles, which can be linked to the porosity, is preferably greater than 10 m²/g, preferably greater than 50 m²/g.

According to one particular embodiment, the “scattering” filler is spherical and porous and has a specific surface area of greater than 10 m²/g, preferably greater than 50 m²/g.

In one preferential embodiment of the invention, the “scattering fillers” are matifying fillers.

### Mattness Test:

The matifying nature of the fillers according to the invention is defined by a gonioreflectometer measurement. To do this, a composition containing 5% of scattering fillers is spread, in a proportion of 2 mg/cm², onto a contrast card (Prufkarte type 24/5-250 cm² sold by the company Erichsen) using a mechanical film spreader. The composition is then dried overnight at a temperature of 37° C., and then the reflection is measured using a gonioreflectometer. The result obtained is the ratio R between the specular reflection and the diffuse reflection. The value of R is proportionately smaller the greater the matifying effect. The matifying scattering fillers according to the invention are those which, at a content of 5% in a cosmetic composition, give a value of R of less than 1.5, and preferably less than 1.

In an alternative, the matifying effect of the fillers are measured according to the following protocole:

The matifying effect of the fillers according to the invention is defined with a measure with the glossmeter. We spread on contrast card (Erichsen type 24/5) a film of 30 μm thickness of a composition containing 3% of “scattering fillers” using a mechanical film spreader. The film is placed at 37° C. for 24 hours, then we measure the brightness with a glossmeter Micro-TR1-gloss from BYK Gardner on the white part on the contrast card, the black portion being to check the homogeneity of the deposit. We measure the specular reflexion of the illuminated film (incidence angle 60°); the measure is then normalised with comparison to a standard and reported to 100 (the more the film is bright, the more the value is close to 100). The matifying scattering fillers of the invention are the ones which, at 3% by weight in the said cosmetic medium, give a brightness value less than 20, preferably less than 10.

More particularly, these scattering, in particular matifying, fillers may, for example, be chosen from:
porous microparticles of silica, for instance the Silica beads SB150 and SB700 from Miyoshi with a mean size of 5 microns; Sunspheres Series-H from Asahi Glass, for instance the Sunspheres H33, H51 and H53 with respective sizes of 3, 5 and 5 μm;
polytetrafluoroethylene powders, for instance the PTFE Ceridust 9205F from Clariant, with a mean size of 8 microns;
silicone resin powders, for instance the silicone resin Tospearl 145A from GE Silicone, with a mean size of 4.5 microns;
hollow hemispherical particles of silicone, for instance NLK 500, NLK 506 and NLK 510 from Takemoto Oil and Fat;
powders of acrylic copolymers, in particular of poly(methyl (meth)acrylate), for instance the PMMA Jurymer MBI particles from Nihon Junyoki, with a mean size of 8 microns, the hollow PMMA spheres sold under the name Covabead LH85 by the company Wackherr, and the isobutane-expanded vinylidene chloride/acrylonitrile/methylene methacrylate microspheres sold under the name Expancel by the company Akzo Nobel;
wax powders, for instance the Paraffin wax microease 114S particles from Micropowders, with a mean size of 7 microns;
polyethylene powders, in particular comprising at least one ethylene/acrylic acid copolymer, and in particular consisting of ethylene/acrylic acid copolymers, for instance the Flobeads EA 209 particles from Sumitomo (with a mean size of 10 microns);
crosslinked elastomeric organopolysiloxane powders coated with silicone resin, in particular with silsesquioxane resin, as described, for example, in U.S. Pat. No. 5,538,793. Such elastomer powders are sold under the names KSP-100, KSP-101, KSP-102, KSP-103, KSP-104 and KSP-105 by the company SHIN-ETSU;
polyamide (Nylon®) powders, for instance the Nylon 12 particles of the Orgasol type from Atofina with a mean size of 10 microns;
the perlites,
and mixtures thereof.

According to a preferred embodiment, the composition comprises at least scattering matifying fillers chosen from:
porous microparticles of silica, for instance the Silica beads SB150 and SB700 from Miyoshi with a mean size of 5 microns; Sunspheres Series-H from Asahi Glass, for instance the Sunspheres H33, H51 and H53 with respective sizes of 3, 5 and 5 μm;
silicone resin powders, for instance the silicone resin Tospearl 145A from GE Silicone, with a mean size of 4.5 microns;
hollow hemispherical particles of silicone, for instance NLK 500, NLK 506 and NLK 510 from Takemoto Oil and Fat;
crosslinked elastomeric organopolysiloxane powders coated with silicone resin, in particular with silsesquioxane resin, as described, for example, in U.S. Pat. No. 5,538,793. Such elastomer powders are sold under the names KSP-100, KSP-101, KSP-102, KSP-103, KSP-104 and KSP-105 by the company SHIN-ETSU;
perlites,
and mixtures thereof.

According to one particular embodiment, the composition of the invention comprises at least “scattering” fillers (ii) chosen from porous microparticles of silica, for example the Silica beads SB700 from Miyoshi; hollow hemispherical particles of silicone, such as the NLK 506 from Takemoto Oil and Fat, and mixtures thereof.

According to one preferred embodiment, the composition of the invention comprises at least porous microparticles of silica, for instance the Silica beads SB700 from Miyoshi.

According to another preferred embodiment, the composition of the invention comprises at least hollow hemispherical particles of silicone, such as the NLK 506 from Takemoto Oil and Fat, and mixtures thereof.

Said “scattering” filler is generally present in the composition in a content ranging from 0.1 to 4% by weight, preferably from 0.5 to 3% by weight, relative to the total weight of said composition.

Advantageously, the composition of the invention comprises at least
(i) titanium oxides, and
(ii) hollow microparticles of silica, and/or hollow hemispherical particles of silicone.

According to one preferred embodiment, the “scattering” fillers (ii) and the “white” pigments (i) mentioned above will be present in the composition of the invention in a “scattering” fillers (ii)/particular pigments (i) weight ratio (by weight of active material) of between 0.1 and 2.5, preferably between 0.5 and 2.5.

### Additional Fillers

The composition may also contain at least one additional filler, intended for improving the application, active-agent transport or comfort properties of the compositions.

Mention may in particular be made of:
particles or microparticles of waxes or which are natural: beeswaxes, jojoba waxes, carnauba waxes, of natural high-melting-point triglycerides in amorphous or crystalline form,
particles of natural or modified natural cellulose, of natural or modified hyaluronic acid,
particles of “gums” or polysaccharides which are natural or of biotechnological origin,
natural or modified microfibers or microparticles (bamboo, cotton, flax, hemp, ramie, abaca, Lyocell®, xylose, starches and modified starches),
amorphous or crystalline particles, such as talcs, silicates (powders of quartz, semi-precious stones, jade, etc.), clays, kaolins, montmorillonites, laponites, saponites, etc.,
particles of guanine, of mica, of talc, of sericite, of shellfish,
composite particles, such as particles of porous silica comprising titanium dioxides, in particular sold under the name God Ball PC-LS by the company Suzuki Oil and Fat. In particular, use will be made of a mixture of titanium oxide and iron oxide that are encapsulated in silica (TiO₂ 44.7%-silica 50%-iron oxide 5.3%), sold under the name PC-LS-14 by MIYOSHI KASEI,
or mixtures thereof.

These additional fillers will be present in the composition in a content ranging from 0 to 3% by weight, in particular from 0.1 to 2% by weight, in particular from 0.2 to 1% by weight, relative to the total weight of said composition.

Those skilled in the art will adjust the amount of total fillers (scattering fillers+additional fillers) such that it remains less than or equal to 4% by weight, relative to the total weight of said composition.

Preferably, the composition of the invention comprises fillers in a total content of less than or equal to 4% by weight, relative to the total weight of said composition, in particular less than or equal to 3% by weight, relative to the total weight of said composition.

### Additional Pigments

The composition of the invention may or may not comprise non-white, coloured additional pigments, such as iron oxides, interference pigments, and mixtures thereof.

According to one particular embodiment, the composition of the invention comprises a content of less than or equal to 0.5% by weight of iron oxides, in particular less than or equal to 0.3% by weight, and better still less than or equal to 0.1% by weight, relative to the total weight of said composition.

The iron oxides are in particular chosen from red, yellow or brown iron oxides and colouring earths or natural ochres which are composite particles that exist naturally, comprising a colouring mineral part (natural pigments) and a non-colouring mineral part (natural fillers).

According to one particular embodiment of the invention, the composition of the invention comprises a content of less than or equal to 1% by weight of composite interference pigments other than the interference white pigments (i) described above, in particular less than 1% by weight, especially less than or equal to 0.5% by weight, in particular less than or equal to 0.1% by weight, or even less than or equal to 0.01% by weight, relative to the total weight of the composition.

According to one particular embodiment, the composition of the invention will not comprise non-white, coloured additional pigments.

According to one particularly preferred embodiment, the composition according to the invention comprises fillers in a total content of less than or equal to 4% by weight, relative to the total weight of said composition, and a content of less than or equal to 0.5% by weight of iron oxides.

More preferably, the composition according to the invention comprises fillers in a total content of less than or equal to 4% by weight, relative to the total weight of said composition, and a content of less than or equal to 0.1% by weight of iron oxides.

According to another embodiment, the composition according to the invention comprises fillers in a total content of less than or equal to 4% by weight, relative to the total weight of said composition, and a content of less than or equal to 1% by weight of composite interference pigments other than the interference white pigments (i) described above.

### Protocol for Measuring the Opacity of the Compositions According to the Invention

The compositions of the invention are in particular characterized by a transparency Tr of between 50 and 70%, as in particular measured according to the protocol described hereinafter.

The transparency of the compositions is measured according to the following protocol:

The composition is spread on a transparent film (Hp Color laser jet transparency, Hp Invent; CP2936A) by means of an automatic applicator from Braive instruments (wet thickness 50 μm). The spreadings are then placed in a thermostated and ventilated oven for 24 hours at 37° C.

Once dried, these films are placed for evaluation on a contrast card (Prufkarte type 24/5-250 cm², sold by the company Erichsen). The opacity is then measured using a Minolta CR-400 colorimeter from the values Y of the black and white parts obtained in the tristimulus system (X, Y, Z).

The transparency value is obtained from the following equation:

[1−(Y black area/Y white area)]×100=% transparency.

If the film is totally opaque, the transparency is equal to 0.

### Protocol for Measuring the Optical Properties of the Compositions According to the Invention

The colour measurements (chroma C, luminance L and hue h), before and after application of a composition according to the invention (onto a transparent film), are carried out on a contrast card representative of the skin of which the lightness is between 30 and 40.

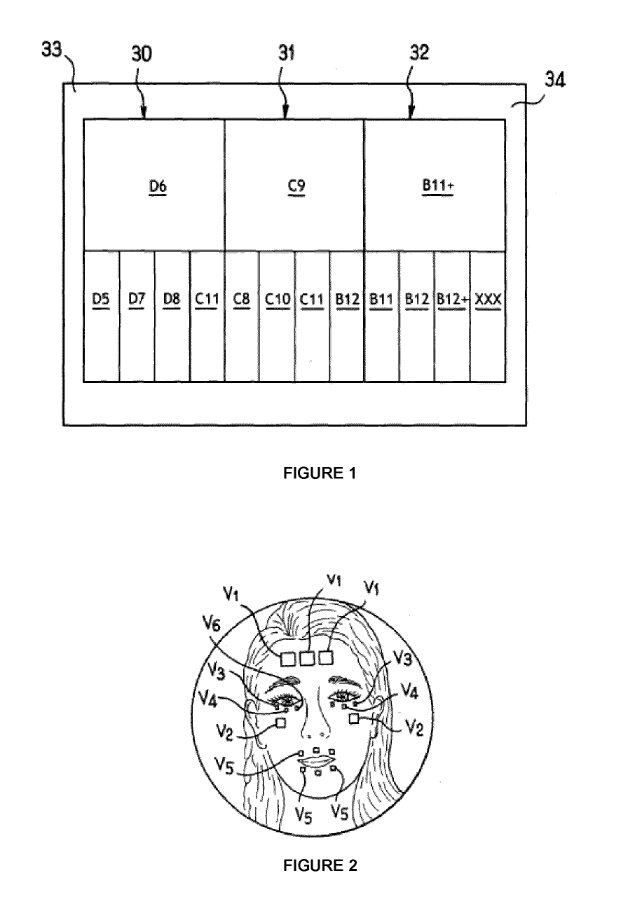

In particular, these measurements are carried out on a contrast card 32 as defined in application EP 1 433 461 and represented in FIG. 4 of said application (reproduced in FIG. 1 of the present application).

Represented on this FIG. 1 are three contrast cards 30, 31 and 32 brought together on the same support 33.

The support 33 comprises a white border 34 which extends all around the contrast cards 30, 31 and 32 which are placed side by side.

The contrast card 32 comprises a square coloured area B11+ and four rectangular coloured areas B11, B12, B12+ and XXX.

The various areas B11, B11+, B12, B12+ and XXX are preferably made by printing with inks chosen such that these areas appear to an observer to be of the same colour under at least two differing illuminants, as described in European patent application EP 1 212 961.

The support 33 is, for example, a matt sheet of paper of 130 g/cm², for inkjet printing.

The contrast card 32 may be representative of skin of which the lightness is between 30 and 40.

Thus, the various areas B11+, B11, B12, B12+ and XXX can correspond to average colours observed on a panel of individuals having dark skin with a dark complexion as regards various regions of the face, represented in FIG. 2 of the present application.

The region B11+ can correspond to the average of the colour measured on the forehead in the areas V1 averaged with that measured on the cheekbones, in the areas V2.

The area B11 can correspond to the colour measured on the cheekbones, in the area V2, the area B12 to that measured on the forehead, in the areas V1, the area B12+ to the average colour between that measured on the outer dark shadows in the areas V3, that measured in the dark shadows in the middle in the areas V4 and that measured around the lips in the areas V5. The area XXX can correspond to the colour of the inner dark shadows, measured in the areas V6.

The contrast card 32 is characterized, for each area, by the following values:

| Area | B11+ | XXX | B12+ | B12 | B11 |
|---|---|---|---|---|---|
| L* | 36.7 | 29.63 | 32.98 | 35.66 | 38.43 |
| C* | 19.81 | 15.06 | 17.29 | 19.78 | 21.76 |
| h | 47.34° | 40.34° | 44.64° | 46.32° | 46.51° |

Where L* is the lightness, and C* the saturation, and h the hue, in the CIE 1976 colorimetric space.

The measurements were carried out with a MINOLTA® spectrocolorimeter, of reference CM2002, in reflection mode, specular included, medium aperture.

The spectral reflectances measured are those given in Table I of application EP 1 433 461, these values being given by way of non-limiting example only.

The values of L* and h are given to within 15%, or even to within 12.5%, better still to within 10%, or even to within 7.5%, or even to within 5%, and the values of C* are given to within 25%, better still to within 20%, even better still to within 15%, even to within 10% or even to within 5%.

In particular, the values of L*, h and C* are given to within 15%.

These colour measurements are carried out using a colorimeter. It is a KONICA MINOLTA CR-400 chromameter. It is a tristimulus colorimeter which contains filters, detectors and a filtered lamp. The measurements are carried out in the colorimetric space: L, C, h. The operating conditions are the following:
Standard observer CIE 1964 10°
Illuminant CIE type D65
Measurement geometry d/0, variant of d/8. The instrument is equipped with an optical device which produces diffuse light, placed in a spherical cavity coated with a white layer which induces a multiple reflection of the light. An anti-glare trap eliminates the effects of glare of the surface of the sample.

Initially, a “white plate” calibration of the instrument is carried out.

The colorimetric parameters L, C and h are then measured on the area B11+ of the bare contrast card 32.

In parallel, the composition to be evaluated is spread on a transparent film (Hp Color laser jet transparency, Hp Invent; CP2936A) by means of an automatic applicator from Braive instruments (wet thickness 50 μm). The spreadings are then placed in a thermostated and ventilated oven for 24 hours at 37° C.

Once dried, these films are placed for evaluation on the area B11+ of the contrast card 32. The parameters ΔC and ΔL are defined as the respective variations in the chroma C and in the luminance L of the area B11+ after application of a cosmetic composition according to the invention.

Once spread into a thin film and dried, the compositions of the invention are characterized by the following optical properties

−25<ΔC<−2 where C is the chroma
5<ΔL<30 where L is the luminance
50<% Tr<70, in particular 50<% Tr<68, or even 50<% Tr<66 where % Tr is the transparency.

### Galenical Forms

The compositions according to the invention are in any of the galenical forms suitable for application to the area around the eyes: suspensions, dispersions, gels, sera, fluid or solid emulsions (W/O, O/W, multiple emulsions), aqueous, anhydrous or fatty sticks, solid, liquid or pasty anhydrous products, loose or compacted powders, cast, moulded, extruded forms.

According to one particular embodiment, the composition is in the form of an emulsion, in particular a fluid or solid oil-in-water emulsion.

In particular, it will be a fluid O/W emulsion.

The term products in “solid” form is intended to mean, according to the invention, a composition having a hardness such that it does not flow at ambient temperature under its own weight, as opposed to compositions that are said to be fluid. Two-phase or multiphase preparations or preparations which are provided in two compartments or multicompartments, one of the phases of which or one of the compartments of which corresponds to the description above, or of which the final mixture of the contents corresponds to the description above.

According to one particular embodiment, the composition according to the invention is in the form of an oil-in-water emulsion, comprising an oily phase dispersed in an aqueous phase, and comprises at least one physiologically acceptable medium.

The term “physiologically acceptable medium” is intended to denote a medium that is particularly suitable for the application of a composition of the invention to keratin materials, in particular the skin and/or the ocular mucous membrane and possible contact with lacrimal fluid.

The physiologically acceptable medium is generally adjusted to the nature of the support onto which the composition must be applied, and also to the way in which the composition must be packaged.

### Aqueous Phase

A composition according to the invention may comprise at least one aqueous phase, preferably in a content ranging from 10 to 99%, and more particularly from 20 to 70% by weight, relative to the total weight of the composition.

The aqueous phase generally comprises water and, optionally, at least one water-soluble solvent.

The water may be a floral water such as cornflower water and/or a water extracted from plants or from fruits and/or a possibly thermal-spring, mineral water such as VITTEL water, LUCAS water or LA ROCHE POSAY water and/or a spring water, a sea water, a fossil water, a melting glacier water, or a distilled water, double-distilled water or water sterilized by any other process.

The term “water-soluble solvent” denotes, in the present invention, a compound that is liquid at ambient temperature and water-miscible (water-miscibility greater than 50% by weight at 25° C. and atmospheric pressure).

The water-soluble solvent(s) suitable for the invention may be chosen from C₁₋₈, and in particular C₁₋₅, monoalcohols, in particular ethanol, isopropanol, tert-butanol and n-butanol, polyols as previously described, and mixtures thereof. Ethanol and isopropanol, and preferably ethanol, are also most particularly suitable for the invention. According to one preferred embodiment, the composition according to the invention comprises at least one C₁-C₄ monoalcohol, in particular ethanol.

A composition of the invention may also comprise at least one salt, for example sodium chloride, magnesium chloride, magnesium sulphate, calcium chloride or calcium phosphate, alone or in the form of a buffer mixture.

A composition of the invention may comprise from 0.05% to 1.5%, in particular from 0.1% to 1.0% by weight of salts relative to the total weight of the composition, and more particularly a concentration which makes it possible to achieve isotonicity, i.e. that corresponding to 0.9% of NaCl, relative to the weight of the aqueous phase.

The aqueous phase may contain components intended to reduce the water activity, such as, for example, polyols, in particular for a water activity <0.9, especially ranging from 0.5 to 0.8.

The aqueous phase may also comprise any water-soluble or water-dispersible compound compatible with an aqueous phase, such as gelling agents, film-forming polymers, thickeners, soluble colorants or coloured molecules, antiseptics such as copper sulphate, surfactants and mixtures thereof, or water-soluble sunscreens.

### Liquid Fatty Phase

A cosmetic composition in accordance with the present invention comprises at least one liquid fatty phase comprising at least one oil.

A composition of the invention may comprise a liquid fatty phase in a content ranging from 1 to 50%, in particular from 2 to 35%, and more particularly from 5 to 25% by weight, relative to the total weight of the composition.

The term “oil” is intended to mean any fatty substance that is in liquid form at ambient temperature (20-25° C.) and at atmospheric pressure.

The oil can be chosen from volatile and non-volatile oils of hydrocarbon-based, silicone or fluoro type. The oils may be of animal, vegetable, mineral or synthetic origin.

The term “volatile oil” is intended to mean an oil (or non-aqueous medium) capable of evaporating on contact with the skin in less than one hour, at ambient temperature and atmospheric pressure. The volatile oil is a volatile cosmetic oil that is liquid at ambient temperature, having in particular a non-zero vapour pressure, at ambient temperature and atmospheric pressure, in particular having a vapour pressure ranging from 0.13 Pa to 40 000 Pa (10⁻³ to 300 mmHg), and preferably ranging from 1.3 Pa to 13 000 Pa (0.01 to 100 mmHg), and preferentially ranging from 1.3 Pa to 1300 Pa (0.01 to 10 mmHg).

In addition, the volatile oil generally has a boiling point, measured at atmospheric pressure, ranging from 150° C. to 260° C., and preferably ranging from 170° C. to 250° C.

Advantageously, the fatty phase comprises from 1 to 30% by weight, preferably ranging from 2 to 20% by weight, and preferentially ranging from 3 to 15% by weight, of volatile oil(s), relative to the total weight of the composition.

The term “hydrocarbon-based oil” is intended to mean an oil formed essentially, or even consisting, of carbon and hydrogen atoms, and optionally of oxygen and nitrogen atoms, and containing no silicon or fluorine atom; it may contain ester, ether, amine and/or amide groups.

The term “silicone oil” is intended to mean an oil containing at least one silicon atom, and in particular containing Si—O groups.

The term “fluoro oil” is intended to mean an oil containing at least one fluorine atom.

### Volatile Oils

The volatile oil may be a hydrocarbon-based or silicone oil. In particular, use will be made of a hydrocarbon-based volatile oil which can be chosen from hydrocarbon-based oils having a flash point ranging from 40° C. to 102° C., preferably ranging from 40° C. to 55° C., and preferentially ranging from 40° C. to 50° C.

As a hydrocarbon-based volatile oil, mention may be made of hydrocarbon-based volatile oils containing from 8 to 16 carbon atoms which are linear or branched, and mixtures thereof, and in particular branched C₈-C₁₆ alkanes, such as C₈-C₁₆ isoalkanes (also called isoparaffins), isododecane, isodecane, isohexadecane, the undecane/tridecane mixture as described in application WO2008/155059, dodecane, tetradecane and mixtures thereof, and for example the oils sold under the trade names Isopar or Permethyl, C₈-C₁₆ branched esters, such as isohexyl neopentanoate, and mixtures thereof. Preferably, the hydrocarbon-based volatile oil is chosen from hydrocarbon-based volatile oils containing from 8 to 16 carbon atoms and mixtures thereof, which are linear or branched.

### Non-Volatile Oils

The non-volatile oil(s) may be present in a content ranging from 0.1% to 20% by weight, relative to the total weight of the emulsion, and preferably ranging from 1% to 15% by weight.

The non-volatile oil may be chosen from carbon-based, hydrocarbon-based and/or silicone and/or fluoro oils of mineral, animal, vegetable or synthetic origin, and mixtures thereof, insofar as they are compatible with the use envisaged.

Mention may be made of non-volatile hydrocarbon-based oils such as liquid paraffin or liquid petroleum jelly, isoeicosane, soya oil, perhydrosqualene, sweet almond oil, beauty-leaf oil, palm oil, grapeseed oil, sesame oil, maize oil, rapeseed oil, sunflower oil, cottonseed oil, apricot oil, castor oil, avocado oil, jojoba oil, olive oil or cereal germ oil; esters of lanolic acid, of oleic acid, of lauric acid, of stearic acid; fatty esters, such as isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate or lactate, 2-diethylhexyl succinate, diisostearyl malate, glyceryl triisostearate or diglyceryl triisostearate; carbonates, such as dicaprylyl carbonate, ethers, such as dicaprylyl ether, higher fatty acids, such as myristic acid, palmitic acid, stearic acid, behenic acid, oleic acid, linoleic acid, linolenic acid or isostearic acid; higher fatty alcohols, such as cetanol, stearyl alcohol or oleyl alcohol, linoleyl or linolenyl alcohol, isostearyl alcohol or octyldodecanol.

The oily phase may also contain any liposoluble or dispersible compound, for instance biologically active oily extracts, such as vitamins A, D, E and K, carotenoids, ceramides, unsaponifiables, essential fatty acids, long-chain fatty acids, essential oils, UV screens, surfactants or colorants.

### Additives

The compositions of the invention may also contain conventional adjuvants, such as colorants, pigments which do not render the formula covering, fragrances, preservatives, surfactants, thickeners, organic or mineral, physical and chemical sunscreens, sequestering agents, antioxidants, insoluble, liposoluble or water-soluble active agents, moisturizers such as polyols and in particular glycerol, pH adjusters (acids or bases), active agents extracted from plants or resulting from biotechnology, mineral active agents, or tensioning agents.

According to one particular embodiment, the composition of the invention also comprises at least one tensioning agent capable of smoothing the skin and reducing wrinkles and fine lines, or even causing them to disappear, immediately.

Generally, this tensioning effect can be described in terms of percentage shrinkage. This percentage shrinkage can be evaluated by means of an in vitro shrinkage test, which consists in bringing a rectangular test piece (10×40 mm) of elastomer having a modulus of about 20 MPa and a thickness of 100 μm into contact with an aqueous solution, at a concentration of less than or equal to 7% by weight, of the tensioning agent tested. After drying for 3-4 h at 22±3° C. and 40±10% relative humidity RH, the tension exerted by this deposit on the substrate and, consequently, the tensioning potential is directly related to the decrease in the width at the centre of the test piece. The tensioning effect (TE) can then be quantified in the following manner:

“TE′”=(L0−L3h/L0)×100 as %
with L0=initial width 10 mm
and L3h=width after 3 h of drying

More specifically, an agent is described as a “tensioning” agent if, when it is used at a concentration by weight of less than or equal to 7%, in particular between 0.5 and 2% by weight, in water or any physiologically acceptable medium, it produces, under such conditions, a shrinkage of at least 10%, preferably at least 15%.

As tensioning agents, mention may in particular be made of:
vegetable proteins and hydrolysates thereof, such as soya proteins, in particular those sold under the names ELESERYL SH/VE G 8 and ELESERYL SH VEG 8B by the company Laboratoires seriobiologiques (INCI name: GLYCINE SOJA SOYBEAN PROTEIN);
colloidal silica particles, such as those sold by the company Catalysts et Chemicals under the names Cosmo S-40 and Cosmo S-50, or silica-alumina composite particles, such as those sold by the company Grace under the names Ludox AM, Ludox AM-X 6021, Ludox HSA and Ludox TMA,
interpenetrating polymers (IPNs), in particular those comprising a polyurethane polymer and a polyacrylic polymer, such as the one prepared according to the process described in U.S. Pat. No. 5,173,526. Preferably, use may be made of an IPN of a polyurethane copolymer of neopentyl glycol/adipic acid/4,4′-methylenebis(cyclohexyl isocyanate)/dimethylolpropanoic acid and of polymethyl methacrylate, such as the product sold by the company AIR PRODUCTS under the trade name Hybridur® 875 polymer dispersion (INCl name: POLYURETHANE-2 (and) POLYMETHYL METHACRYLATE), or alternatively under the trade names Hybridur® 870 and Hybridur® 880;
grafted silicone polymers, such as: a propylthio(polymethyl acrylate/methyl methacrylate/methacrylic acid)-grafted polydimethylsiloxane, a propylthio(polymethyl acrylate)-, propylthio(polymethyl methacrylate)- and propylthio(polymethacrylic acid)-grafted polydimethylsiloxane, or a propylthio(polyisobutyl methacrylate)- and propylthio(polymethacrylic acid)-grafted polydimethylsiloxane. Use is preferably made of a propylthio(polymethyl acrylate/methyl methacrylate/methacrylic acid)-grafted polydimethylsiloxane, such as the product available under the trade name VS 80 or VS 70 (at 10% in water) or LO 21 (in pulverulent form) from the company 3M.

These additives are generally used at concentrations ranging from 0.1% to 10% of the total weight of the final composition.

### Active Agents

According to one particular embodiment, use will be made of active agents chosen from agents for promoting the microcirculation or reinforcing the walls of blood or lymphatic vessels, anti-pigmenting or depigmenting agents, calmatives, vitamins, antioxidants, and mixtures thereof, active agents for toning and firming skin structures or increasing their fibrillar or matricial cell density, or opposing their degradation, active agents for acting on lipolysis or, on the contrary, lipogenesis of adipocytes, anti-wrinkle active agents and moisturizing active agents.

By way of examples, mention may be made of: active agents known for their calming or anti-inflammatory activity, such as extracts of liquorice, of rose, of cornflower, of camomile, olive hydroxytyrosol; anti-wrinkle agents, such as C-β-2 glucopyranoside, adenosine, relaxing active agents; cell energy stimulating agents (amino acids, oligopeptides, RNA derivatives or DNA derivatives, coenzymes [Q10], trace elements; free-radical scavengers; vascular protective or toning agents (ginkgo derivatives, red vine leaf extracts, pine or grapeseed OPC oligomeric proanthocyanidins, extracts of fruits, berries, petals, barks, seeds rich in anthocyans, such as, for example, blueberry extract (WO01/45648); agents for promoting blood or lymphatic microcirculation (xanthine bases, caffeine, theophilline, theobromine and derivatives, extracts of coffee, of tea, of cocoa, of guarana, nicotinic derivatives at a dose compatible with good innocuousness); immunity-protecting agents or antibacterial protective agents, honey or hive-derived products, extracts, lysates or lyophilisates of microorganisms such as yeasts or probiotics, saline solutions and artificial tears, anti-glycation or glycoxidation agents. Mention may also be made of vitamins, such as, for example, vitamins A, C, D, B3, B6, or PP, B5, E, K1 or K2, and precursors or derivatives of these vitamins, and in particular esters thereof or a combination thereof with sugars; carotenes and carotenoids, such as, for example, beta-carotene, lutein, astaxanthin or lycopene, which can be in the form of nanocapsules; antioxidants and in particular natural antioxidants derived from plants or from biotechnology, polyphenols, sunscreens or UV-screening particles; moisturizers such as polyols; ceramides; DHEA and derivatives thereof; whitening and depigmenting agents, such as kojic acid, vitamin C and its derivatives, para-aminophenol derivatives, arbutin and their derivatives, tensioning or smoothing agents such as soy hydrolysates, and mixtures thereof.

According to one particular embodiment, the composition according to the invention does not comprise any whitening active agent.

According to one particular embodiment, the composition comprises at least one active agent for acting on the microcirculation, intended for improving the appearance of the area around the eye, in particular reducing the dark circles. It may be chosen, for example, from
flavonoids and glucosides thereof, such as ginkgo flavoglycosides, amentoflavone or dimeric flavones of Gingko biloba, citroflavonoids, hesperidin, alpha-G-hesperidin, hesperidin methyl chalcone, rutin and rutosides (for example: beta-hydroxyethyl-rutoside, trimethyl-rutoside);
saponosides, such as escin, escin beta-sitosterol, ruscogenin and neoruscogenin, asiaticoside and madecassoside, diosgenin,
coumarins such as esculoside, esculetol or methylesculetol,
vegetable extracts, such as extract of ruscus, of horse chestnut, of ivy, of coffee, of green tea or black tea (fermented), of cocoa, extract of everlasting from Corsica (Helichrysum italicum) as described, in particular, in application WO 03/018730; extracts of blackcurrant (Ribes nigrum), of fruits of the genus Vaccinium (bilberries: V. vitis-idaea subsp. vitis-idaea, cranberries: V. macrocarpon, blueberries: V. myrtillus), of the fruit of elder Sambucus nigra or S. racemosa, of fruits of the genus Rubus (raspberries Rubus idaeus), of Myrica rubra, of mistletoe, of kiwi (Actinidia chinensis L.), of cypress (Cupressus sempervirens), of melilot (Melissa officinalis), of ash, of mouse-ear hawkweed, of tonka bean, of lesser periwinkle (Vinca minora), of hydrocotyl (Centella asiatica), of Terminalia sericea (sericoside), extracts of Calendulae, extracts of Arnica, extracts of Ammi visnaga; extracts of angelica, of coriander, of liquorice, of Saphora japonica, of Eucalyptus macrorryncha, of witch-hazel, of Peruvian rhatany, of lesser celandine, of agave, of yucca, of Dioscoreaceae, of Bupleurum, of safflower, of the roots of plants of the genus Astragalus, of Salvia milthiorrhizae, of Paeonia rubra, of the rhizome of Polygala senega, of a fermented preparation such as, for example, kombucha originating from fermented tea,
other vasodilators, such as nicotinic acid (niacin) and derivatives thereof, for instance nicotinic acid esters, for example xanthinol nicotinate, inositol nicotinate; methyl, ethyl or hexyl nicotinate,
essential oils of lavender, of thyme, of rosemary, of hesperidia, of mint, of eucalyptus, of geranium, of rose,
adenosine or ATP (adenosine triphosphate);
and mixtures thereof.

In particular, use will be made of an extract of Gingko biloba, an extract of centella asiatica, of tea, of blackcurrant, of blueberry, of rose, caffeine, or mixtures thereof.

These compounds are generally used at concentrations ranging from 0.1% to 10% of the total weight of the final composition.

FIG. 1 represents contrast cards (in particular contrast card 32) used for determining the colorimetric characteristics of the compositions of the invention.

FIG. 2 represents diagrammatically the areas of the face on which the colorimetric parameters are measured.

The invention will now be illustrated by means of the following non-limiting examples. Unless otherwise indicated, the values of the ingredients in the compositions are expressed as % by weight, relative to the total weight of said composition.

### EXAMPLES

The following compositions were prepared:

### Example 1

### W/O Emulsion

| INGREDIENTS | % | Phase |
|---|---|---|
| GLYCEROL | 5 | A |
| CAPRYLYL GLYCOL | 0.5 | A |
| METHYLPARABEN | 0.25 | A |
| DISODIUM EDTA | 0.15 | A |
| GINKGO BILOBA LEAF EXTRACT | 0.1 | A |
| (from LINNEA) | | |
| DEIONIZED WATER | qs 100 | A |
| DIMETHICONE (and) DIMETHICONE/ | 3.6 | B1 |
| PEG-10/15 CROSSPOLYMER KSG 210 | | |
| from Shin Etsu | | |
| SILICA DIMETHYL SILYLATE (and) | 1 | B1 |
| SODIUM HYALURONATE | | |
| SYNTHETIC WAX Cirebelle 303 | 2 | B1 |
| from Cirebelle | | |
| PEG-10 DIMETHICONE KF-6017 | 0.5 | B1 |
| from Shin Etsu | | |
| ISOHEXADECANE | 3.4 | B1 |
| PROPYLENE CARBONATE | 0.16 | B1 |
| DISTEARDIMONIUM HECTORITE | 0.6 | B2 |
| BENTONE 38 VCG from Elementis | | |
| RUTILE TITANIUM OXIDE TREATED | 0.75 | B3 |
| WITH ALUMINA/SILICA/TRIMETHYLOL- | | |
| PROPANE (CI: 77891) (TIPAQUE PF-671 | | |
| (COSMETIC GRADE) from ISHIHARA | | |
| SANGYO) “pigment” | | |
| DIMETHICONE | 4 | B3 |
| SODIUM POLYACRYLATE COSMEDIA | 1 | C |
| SP from Cognis | | |
| DIMETHICONE | 1 | C |
| METHYLSILANOL/SILICATE | 0.75 | D |
| CROSSPOLYMER (NLK 506 | | |
| FROM TAKEMOTO OIL & FAT) | | |
| “SCATTERING FILLER” | | |
| TITANIUM DIOXIDE AND IRON | 2 | D |
| OXIDE ENCAPSULATED IN POROUS | | |
| SILICA (44.7% TiO₂, 50% | | |
| silica, 5.3% iron oxide, PC-LS-14 | | |
| FROM MIYOSHI KASEI) | | |

### Procedure:

phase B is heated to approximately 75° C. and the various ingredients are incorporated therein and the mixture is stirred until a homogenous gel is obtained.
phase A is heated to approximately 75° C.
the emulsion is prepared by incorporating phase A into phase B.
the remaining phases are incorporated at 40-45° C. and the stirring is maintained until complete cooling.

### Example 2

### O/W Emulsion

| INGREDIENTS | % | PHASE |
|---|---|---|
| GLYCEROL | 4 | A1 |
| 1,2-OCTANEDIOL | 0.4 | |
| PROPANEDIOL | 3 | |
| 2-PHENOXYETHANOL | 0.6 | |
| SODIUM BENZOATE | 0.125 | |
| OXYETHYLENATED (16 EO) DIMETHYLSILOXANE WITH METHOXY ENDS | 1.25 | |
| (DOW CORNING 2501 COSMETIC WAX FROM DOW CORNING) | | |
| ETHYLENEDIAMINETETRAACETIC ACID, DISODIUM SALT, 2 H₂O | 0.075 | |
| DIPROPYLENE GLYCOL | 3.5 | |
| CENTELLA ASIATICA EXTRACT | 1 | |
| CAFFEINE | 1.5 | |
| DEIONIZED WATER | QS 100 | |
| OXYETHYLENATED (20 EO) METHYL GLUCOSIDE SESQUISTEARATE | 0.5 | A2 |
| (GLUCAMATE SSE-20 EMULSIFIER FROM LUBRIZOL) | | |
| MIXTURE OF ETHOXYLATED (15 EO) SODIUM ETHYLDIAMINO-N- | 2.5 | B1 |
| COCOYL SULPHONATE/BEHENYL ALCOHOL/GLYCERYL | | |
| STEARATE/GLYCERYL CITRATE (CERALUTION H FROM SASOL) | | |
| TOCOPHEROL | 0.1 | |
| 2-OCTYLDODECANOL | 1 | |
| POLY(STEARYL ACRYLATE) (INTELIMER IPA 13-1 FROM AIR PRODUCTS | 0.5 | |
| AND CHEMICALS) | | |
| NON-STABILIZED, REFINED, DEODORIZED APRICOT KERNEL OIL | 1.5 | B2 |
| (TRIGLYCERIDES OF OLEIC-LINOLEIC ACIDS 66/28) | | |
| RUTILE TITANIUM OXIDE TREATED WITH | 0.75 | B3 |
| ALUMINA/SILICA/TRIMETHYLOLPROPANE (CI: 77891) (TIPAQUE PF-671 | | |
| (COSMETIC GRADE) FROM ISHIHARA SANGYO) “PIGMENT” | | |
| DIISOPROPYL SEBACATE | 2 | B3 |
| ISONONYL ISONONANOATE | 1 | C1 |
| MIXTURE OF ALPHA,OMEGA-DIHYDROXY POLYDIMETHYL- | 2.5 | |
| SILOXANE/POLYDIMETHYLSILOXANE 5 CST (DOW CORNING 1503 FLUID | | |
| FROM DOW CORNING) | | |
| N-UNDECANE:N-TRIDECANE MIXTURE IN WHICH THE N-UNDECANE IS IN | 5 | |
| THE MAJORITY IN THE MIXTURE AS PREPARED ACCORDING TO | | |
| APPLICATION WO2008/155059 | | |
| HIGHLY CROSSLINKED POLYACRYLAMIDOMETHYLPROPANESULPHONIC | 0.9 | |
| ACID PARTIALLY NEUTRALIZED WITH AQUEOUS AMMONIA | | |
| (HOSTACERIN AMPS FROM CLARIANT) | | |
| XANTHAN GUM | 0.1375 | |
| ACRYLAMIDE/SODIUM ACRYLAMIDO-2-METHYLPROPANESULPHONATE | 0.125 | C2 |
| COPOLYMER IN AN INVERSE EMULSION AT 40% IN ISOPARAFFIN/WATER | | |
| (SEPIGEL 305 FROM SEPPIC) | | |
| DENATURED ABSOLUTE ETHYL ALCOHOL | 2.95 | C3 |
| HIGH-MOLECULAR-WEIGHT [POLYDIMETHYLSILOXANE] [ETHYL] | 0.25 | |
| [POLYDIMETHYLSILOXANE] BLOCK COPOLYMER, IN AN AQUEOUS | | |
| EMULSION AT 61%, PROTECTED (DOW CORNING HMW 2220 NON-IONIC | | |
| EMULSION FROM DOW CORNING) | | |
| FRAGRANCE | 0.2 | C4 |
| METHYLSILANOL/SILICATE CROSSPOLYMER (NLK506 TAKEMOTO FAT & OIL) | 0.75 | D |
| “SCATTERING FILLER” | | |
| TITANIUM DIOXIDE AND IRON OXIDE ENCAPSULATED IN POROUS | 1 | |
| SILICA (44.7% TiO₂, 50% silica, 5.3% iron oxide, PC-LS-14 FROM MIYOSHI | | |
| KASEI) | | |

### Procedure:

phase B is heated to approximately 75° C. and the various ingredients are incorporated therein; the mixture is stirred until a homogeneous gel is obtained.
phase A is heated to approximately 75° C.
the emulsion is prepared by incorporating phase B into phase A.
the remaining phases are incorporated at 40-45° C. and the stirring is maintained until complete cooling.

### Measurements of Opacity and of the Optical Properties of the Compositions of the Invention

The protocols as described above are used.

The following table summarizes the results obtained with Examples 1 and 2.

| | ΔC | ΔL | % Tr |
|---|---|---|---|
| Example 1 | −12 | 25 | 62 |
| Example 2 | −15 | 24 | 65 |

The compositions of the invention are applied to the area around the eye and make it possible to significantly reduce the visibility of the dark circles.

### In Vivo Evaluation of the Optical Properties

The composition of Example 2 was applied to a panel of 6 individuals with dark circles, at the level of the region of the area around the eye (0.025 ml applied). A beautician then evaluates “blind” (the compositions applied are coded and the order of the before/after photos is randomized) the reduction in visibility of the dark circles provided by the composition, compared with bare skin.

Results: A lightening effect of the area around the eye, which reduces the visibility of the dark circles, was noted on all the models. The same test was reproduced on a panel of Chinese women with dark circles. The same results were noted. The combination of a white pigment (i) and a scattering filler (ii) according to the invention therefore makes it possible to visibly decrease the dark circles while keeping a natural “bare skin” appearance.

### Example 3

### O/W Emulsion

| INGREDIENTS | % | PHASE |
|---|---|---|
| GLYCEROL | 7 | A |
| 1,2-OCTANEDIOL | 0.4 | |
| 2-PHENOXYETHANOL | 0.6 | |
| SODIUM BENZOATE | 0.25 | |
| OXYETHYLENATED (16 EO) DIMETHYLSILOXANE WITH | 2.5 | |
| METHOXY ENDS (DOW CORNING 2501 COSMETIC WAX | | |
| FROM DOW CORNING) | | |
| ETHYLENEDIAMINETETRAACETIC ACID, DISODIUM | 0.1 | |
| SALT, 2 H₂O | | |
| DEIONIZED WATER | QS 100 | |
| DRY AQUEOUS-ALCOHOLIC EXTRACT OF RUSCUS | 0.1 | A |
| CONTAINING 10% SAPONINS (7242000 BUTCHERS | | |
| BROOM D.H.E. 9-11% PHYT from BERNETT) | | |
| MIXTURE OF ETHOXYLATED (150 EO) SODIUM | 5 | B1 |
| ETHYLDIAMIDO-N-COCOYLSULPHONATE/BEHENYL | | |
| ALCOHOL/GLYCERYL STEARATE/GLYCERYL CITRATE | | |
| (CERALUTION H FROM SASOL) | | |
| TOCOPHEROL | 0.2 | |
| 2-OCTYLDODECANOL | 2 | |
| POLY(STEARYL ACRYLATE) (INTELIMER IPA 13-1 FROM | 1 | |
| AIR PRODUCTS AND CHEMICALS) | | |
| DIISOPROPYL SEBACATE | 4 | |
| NON-STABILIZED, REFINED, DEODORIZED APRICOT | 3 | B2 |
| KERNEL OIL (TRIGLYCERIDES OF OLEIC-LINOLEIC | | |
| ACIDS 66/28) | | |
| ISONONYL ISONONANOATE | 2 | C1 |
| MIXTURE OF ALPHA,OMEGA-DIHYDROXY | 3 | |
| POLYDIMETHYLSILOXANE/POLYDIMETHYLSILOXANE 5 | | |
| CST (DOW CORNING 1503 FLUID FROM DOW CORNING) | | |
| HIGHLY CROSSLINKED POLYACRYLAMIDOMETHYL- | 1 | |
| PROPANESULPHONIC ACID PARTIALLY NEUTRALIZED | | |
| WITH AQUEOUS AMMONIA (HOSTACERIN AMPS FROM | | |
| CLARIANT) | | |
| XANTHAN GUM | 0.25 | |
| DISPERSION OF BISMUTH OXYCHLORIDE IN | 2 | |
| 2-ETHYLHEXYL HYDROXYSTEARATE 70:30 (TIMIRON | | |
| LIQUID SILVER ®) FROM MERCK “PIGMENT” | | |
| COPOLYMER OF ACRYLAMIDE/SODIUM ACRYLAMIDO-2- | 0.25 | C2 |
| METHYLPROPANESULPHONATE IN AN INVERSE | | |
| EMULSION AT 40% IN ISOPARAFFIN/WATER (SEPIGEL | | |
| 305 FROM SEPPIC) | | |
| FRAGRANCE | 0.3 | C3 |
| SILICA MICROSPHERES (SB 700 FROM MIYOSHI KASEI) | 1 | D |
| “SCATTERING FILLER” | | |
| TITANIUM DIOXIDE AND IRON OXIDE ENCAPSULATED | 1 | |
| IN POROUS SILICA (44.7% TiO₂, 50% silica, 5.3% iron | | |
| oxide, PC-LS-14 FROM MIYOSHI KASEI) | | |

### Procedure:

phase B is heated to approximately 75° C. and the various ingredients are incorporated therein; the mixture is stirred until a homogeneous gel is obtained.
phase A is heated to approximately 75° C.
the emulsion is prepared by incorporating phase B into phase A.
the remaining phases are incorporated at 40-45° C. and the stirring is maintained until complete cooling.

### Example 4

### O/W Emulsion

| INGREDIENTS | % | PHASE |
|---|---|---|
| GLYCEROL | 7 | A |
| 1,2-OCTANEDIOL | 0.4 | |
| 2-PHENOXYETHANOL | 0.6 | |
| SODIUM BENZOATE | 0.25 | |
| OXYETHYLENATED (16 EO) DIMETHYLSILOXANE | 2.5 | |
| WITH METHOXY ENDS (DOW CORNING 2501 | | |
| COSMETIC WAX FROM DOW CORNING) | | |
| ETHYLENEDIAMINETETRAACETIC ACID, DISODIUM | 0.1 | |
| SALT, 2 H₂O | | |
| DEIONIZED WATER | QS 100 | |
| DRY AQUEOUS-ALCOHOLIC EXTRACT OF RUSCUS | 0.1 | A |
| CONTAINING 10% SAPONINS (7242000 BUTCHERS | | |
| BROOM D.H.E. 9-11% PHYT from BERNETT) | | |
| MIXTURE OF ETHOXYLATED (15 EO) SODIUM | 5 | B1 |
| ETHYLDIAMIDO-N-COCOYLSULPHONATE/BEHENYL | | |
| ALCOHOL/GLYCERYL STEARATE/GLYCERYL | | |
| CITRATE (CERALUTION H FROM SASOL) | | |
| TOCOPHEROL | 0.2 | |
| 2-OCTYLDODECANOL | 2 | |
| POLY(STEARYL ACRYLATE) (INTELIMER IPA 13-1 | 1 | |
| FROM AIR PRODUCTS AND CHEMICALS) | | |
| DIISOPROPYL SEBACATE | 4 | |
| NON-STABILIZED, REFINED, DEODORIZED | 3 | B2 |
| APRICOT KERNEL OIL (TRIGLYCERIDES OF OLEIC- | | |
| LINOLEIC ACIDS 66/28) | | |
| ISONONYL ISONONANOATE | 2 | C1 |
| MIXTURE OF ALPHA,OMEGA-DIHYDROXY- | 3 | |
| POLYDIMETHYLSILOXANE/POLYDIMETHYLSILOXANE | | |
| 5 CST (DOW CORNING 1503 FLUID FROM DOW CORNING) | | |
| HIGHLY CROSSLINKED POLYACRYLAMIDOMETHYL- | 1 | |
| PROPANESULPHONIC ACID PARTIALLY | | |
| NEUTRALIZED WITH AQUEOUS AMMONIA | | |
| (HOSTACERIN AMPS FROM, CLARIANT) | | |
| XANTHAN GUM | 0.25 | |
| FLUORPHLOGOPITE COATED WITH TITANIUM OXIDE | 2 | |
| AND WITH TIN OXIDE (SYNCRISTAL SILVER FROM | | |
| ECKART) “PIGMENT” | | |
| COPOLYMER OF ACRYLAMIDE/SODIUM | 0.25 | C2 |
| ACRYLAMIDO-2-METHYLPROPANESULPHONATE IN | | |
| AN INVERSE EMULSION AT 40% IN | | |
| ISOPARAFFIN/WATER (SEPIGEL 305 FROM SEPPIC) | | |
| FRAGRANCE | 0.3 | C3 |
| SILICA MICROSPHERES (SB 700 FROM MIYOSHI | 1 | D |
| KASEI) “SCATTERING FILLER” | | |
| TITANIUM DIOXIDE AND IRON OXIDE | 1 | |
| ENCAPSULATED IN POROUS SILICA (44.7% TiO₂, | | |
| 50% silica, 5.3% iron oxide, PC-LS-14 FROM MIYOSHI | | |
| KASEI) | | |

### Procedure:

phase B is heated to approximately 75° C. and the various ingredients are incorporated therein; the mixture is stirred until a homogeneous gel is obtained.
phase A is heated to approximately 75° C.
the emulsion is prepared by incorporating phase B into phase A.
the remaining phases are incorporated at 40-45° C. and the stirring is maintained until complete cooling.

The compositions of the invention are applied to the area around the eye and make it possible to significantly reduce the visibility of the dark circles and of the local aesthetic defects while keeping a natural “bare skin” appearance.

1. A cosmetic composition comprising, in a physiologically acceptable medium,
(i) a pigment in a form of a porous or non-porous, spherical or non-spherical, mineral or organic white particle having a refractive index of greater than or equal to 1.6 and a size by volume of less than 30 μm, and
(ii) at least one matifying filler in a form of a porous or non-porous, spherical or non-spherical particle having a refractive index of less than or equal to 2 and a size by volume of less than 30 μm, which is distinct from the pigment (i),
wherein a total content of filler is less than or equal to 4% by weight, relative to a total weight of the composition.

2. A cosmetic composition comprising, in a physiologically acceptable medium,
(i) a pigment in a form of a porous or non-porous, spherical or non-spherical, mineral or organic white particle having a refractive index of greater than or equal to 1.6 and a size by volume of less than 30 μm, and
(ii) a filler in a form of a porous or non-porous, spherical or non-spherical particle having a refractive index of less than or equal to 2 and a size by volume of less than 30 μm, which is distinct from the pigment (i),
wherein the composition has optical properties such that:
−25<ΔC<−2 where C is chroma
5<ΔL<30 where L is luminance
50<% Tr<70 where % Tr is transparency
measured on a contrast card representative of skin having a lightness between 30 and 40.

3. The composition of claim 1, wherein the pigment (i) is at least one selected from the group consisting of a titanium oxide, a zinc oxide, an optionally dispersed bismuth oxychloride, an insoluble barium salt, barium sulfate, calcium carbonate, calcium sulfate, a plaster, a natural powder of mineral, animal or vegetable origin, a chalk powder, a gypsum powder, an alabaster powder, a selenite powder, an egg-shell powder, a shellfish powder, and a vegetable ivory.

5. The composition of claim 1, wherein the pigment (i) is a coated or uncoated titanium oxide of rutile or anatase form.

6. The composition of claim 1, wherein the pigment (i) is an optionally dispersed bismuth oxychloride.

7. The composition of claim 1, wherein the pigment (i) is a white-tinted interference pigment.

8. The composition of claim 1, wherein the pigment (i) is present in a content ranging from 0.1% to 4% by weight, relative to the total weight of the composition.

9. The composition of claim 1, wherein the filler (ii) is spherical and porous and has a specific surface area of greater than 10 m²/g.

10. The composition of claim 1, wherein the filler (ii) is at least one selected from the group consisting of:
a porous microparticle of silica,
a silicone resin powder,
a hollow hemispherical particle of silicone,
a crosslinked elastomeric organopolysiloxane powder coated with silicone resin, and
a perlite.

11. The composition of claim 1, wherein the filler (ii) is present in the composition in a content ranging from 0.1 to 4% by weight relative to the total weight of said composition.

12. The composition of claim 1, further comprising less than or equal to 0.5% by weight of an iron oxide, relative to the total weight of the composition.

13. The composition of claim 1, in a form of an emulsion.

14. A cosmetic device comprising
a) a reservoir comprising the composition of claim 1, and
b) an applicator selected from the group consisting of a brush, a perforated flexible blade, a felt tip, a sponge, a roller, a ball, a plate bearing several balls, a pad, and a head capable of generating physical pulses that are mechanical thermal, electrical, electromagnetic, or ultrasonic.

15. A cosmetic process for caring for keratin materials, making up keratin materials, or both, the process comprising applying the composition of claim 1 to skin.

16. The cosmetic process of claim 15, comprising applying the composition to an area around an eye.

17. The composition of claim 1, wherein the pigment (i) is present in a content ranging from 0.5% to 3% by weight, relative to the total weight of the composition.

18. The composition of claim 1, wherein the filler (ii) is spherical and porous and has a specific surface area of greater than 50 m²/g.

19. The composition of claim 1, wherein the filler (ii) is present in the composition in a content ranging from 0.5 to 3% by weight, relative to the total weight of said composition.

20. The composition of claim 1, further comprising less than or equal to 0.1% by weight of an iron oxide, relative to the total weight of the composition
